(19) United States
(12) Patent Application Publication
(10) Pub. No.: US 2026/0124051 A1
(43) Pub. Date: May 7, 2026

(54) PROCESS AND APPARATUS FOR PRODUCING A HOLLOW CYLINDER FOR A MEDICAL APPLICATION

(71) Applicant: BIOTRONIK AG, Buelach (CH)

(72) Inventor: Alexander Heuss, Insel Poel (DE)

(21) Appl. No.: 19/122,809

(22) PCT Filed: Nov. 7, 2023
(86) PCT No.: PCT/EP2023/081006

§ 371 Date: Apr. 21, 2025

(30) Foreign Application Priority Data
Dec. 8, 2022 (EP) 22212143.6

(51) Int. Cl. A61F 2/82; B21C 1/24; B21C 23/08

(52) U.S. Cl. CPC A61F 2/82; B21C 1/24; B21C 23/085; A61F 2230/0069; A61F 2240/001

(57) ABSTRACT

A process for producing a hollow cylinder for an implant includes extruding a blank that is at least partially inserted into a recipient chamber of a recipient by pressing a punch through a die or by pressing both the punch and the die both into the recipient. A mandrel is drive into the blank to form a hollow cylinder with an inside diameter defined by the mandrel and an outside diameter defined by the die. The hollow cylinder is guided through a roll gap defined by at least two rollers.

### PRIORITY CLAIM

This application is a 35 U.S.C. 371 US National Phase and claims priority under 35 U.S.C. § 119, 35 U.S.C. 365 (b) and all applicable statutes and treaties from prior PCT Application PCT/EP2023/081006, which was filed Nov. 7, 2023, which application claimed priority from EP Application Serial No. 22212143.6, which was filed Dec. 8, 2022.

### FIELD OF THE INVENTION

A field of the invention concerns devices and processes for manufacturing medical implants including a hollow cylinder.

### BACKGROUND

Workpieces or semi-finished products in the form of hollow cylinders are used in many areas of medical technology. Stents for keeping hollow orifices or vessels open are an important example. Stents have a body in the form of a tubular or hollow-cylindrical basic grid which is open at both longitudinal ends. Often, hollow-cylindrical semi-finished products are used as the initial shape for such a body, from which the basic grid is then cut out, for example by a laser.

Stents are used in blood vessels, especially in the coronary arteries, to prevent them from being blocked again after they have been dilated; this type of treatment is known as stent angioplasty. On the other hand, stents are used in cancer treatment to keep open constrictions of the airways (trachea), bile ducts or esophagus caused by malignant tumors. In addition to the primary goal of preventing a new vessel occlusion, stents can also fix vascular deposits and improve the blood flow in the vessel, as they smoothen the surface of the vessel interior by being pressed against the vessel wall.

In principle, the required workpieces or semi-finished products in the form of hollow cylinders can be produced by forming processes. One possible method is extrusion or impact extrusion, which is a well-known manufacturing process for thin-walled tubes. In this process, a drilled blank is placed on a mandrel of a punch, which acts as an inner tool and thus defines the final inner diameter of the hollow cylinder, and is then pressed through a die, acting as the outer tool for defining the cylinder's outer diameter. Comparatively high degrees of forming can be achieved in a single process step, allowing to realize good mechanical properties, i.e., small grains, high strength and moderate elongation at break. However, the high degree of forming worsens the resulting cylinder geometry, i.e., continuity of the outer diameter or wall thickness.

In medical applications, the pressing process is often additionally supported by a traction or traction/torsion device. In this case, the device grips the hollow cylinder at the tool outlet and pulls or pulls and twists the hollow cylinder with high forces and/or torsional moments. Depending on the level of the stress that occurs, the microstructure is further improved for the medical application, but the geometry is further deteriorated.

Alternatively, pure drawing processes are used. In this process, a blank is drawn over a mandrel, often a pin (also called free-floating mandrel), in several consecutive production steps. Between the manufacturing steps, a recrystallisation-annealing process takes place to restore the formability of the tube for the subsequent step. Due to the many steps, a very good geometry is achieved but this is achieved at the expense of the mechanical properties as grain growth occurs due to the many intermediate annealing steps.

In summary, extrusion or impact extrusion with or without a tension or tension/torsion device has the disadvantage that the resulting geometric properties of the hollow cylinder are not ideal. In particular, there are comparatively large variations in the outer diameter and wall thickness. Drawing, on the other hand, leads to a coarser structure with large grains and thus, poorer mechanical properties. In addition, a multitude of forming steps, often 10 or more, are necessary to form the final hollow cylinder.

### SUMMARY OF THE INVENTION

A process for producing a hollow cylinder for an implant includes extruding a blank that is at least partially inserted into a recipient chamber of a recipient by pressing a punch through a die or by pressing both the punch and the die both into the recipient. A mandrel is drive into the blank to form a hollow cylinder with an inside diameter defined by the mandrel and an outside diameter defined by the die. The hollow cylinder is guided through a roll gap defined by at least two rollers.

An apparatus for producing a hollow cylinder for an implant includes an extrusion press device including a recipient with a recipient chamber configured to receive a blank. A punch is configured to be guided in the recipient chamber. A press disc is on the punch. A mandrel is on the press disc and is configured to form an inner diameter of the hollow cylinder. A die is configured to form an outer diameter of the hollow cylinder. At least two rollers are downstream of the extrusion press device. The at least two rollers delimit a rolling gap configured to guide the hollow cylinder.

### BRIEF DESCRIPTION OF THE DRAWINGS

FIG. 1 a schematic view of an apparatus according to the invention.

### DETAILED DESCRIPTION OF THE PREFERRED EMBODIMENTS

A preferred process for producing a hollow cylinder for an implant includes extruding a blank that is at least partially inserted into a recipient chamber of a recipient by pressing a punch through a die or by pressing both the punch and the die both into the recipient. A mandrel is drive into the blank to form a hollow cylinder with an inside diameter defined by the mandrel and an outside diameter defined by the die. The hollow cylinder is guided through a roll gap defined by at least two rollers.

A preferred method according to the invention for producing a semi-finished product for medical technology or for a medical product, in particular for an implant, includes the following steps:

In step (i), direct or indirect extrusion is possible. In direct extrusion, the material block, a so-called blank, is inserted into the surrounding recipient and pressed through the die with the help of a punch. This process variant is characterized by the fact that the recipient and the die are stationary.

The indirect variant of extrusion is characterized by the fact that the punch together with the die is pressed into the recipient. This process produces less frictional forces, as there is no relative movement between the punch and the recipient. The efficiency of the pressing movement is therefore higher than with the direct extrusion process. Moreover, there is also no relative movement between the blank and the recipient facilitating the use of longer blanks.

It is possible to foresee a drill hole in the blank for a more accurate positioning of the mandrel. The drill hole can be designed as a blind hole or as a complete opening. It is possible to first join the blank and the punch by the mandrel and then insert them into the recipient chamber of the recipient or to first insert the blank into the recipient chamber and then insert the mandrel and the punch.

However, the mandrel must have a length that is greater than the length of the resulting hollow cylinder. In a first variant, the mandrel can be guided through the punch and driven into the blank independently of its movement in such a way that it penetrates it completely, thus creating a hollow cylinder. In a second variant, the mandrel is attached to the punch and already penetrates the blank completely during insertion, which requires a tubular design of the blank, so a complete opening is necessary.

In all variants, a hollow cylinder is built with an inner diameter formed by the mandrel and an outer diameter formed by the die. The hollow cylinder emerging from the die in the feed direction is then guided through a roll gap defined by at least two rollers in a step (ii) following step (i).

This has the advantage that a tensile force or torsional moment can be applied by the rollers. By this, residual stresses are introduced into the material, recrystallisation is favored and the grain orientation is shaped.

One way to conduct the method is applying tensile forces between 20 to 100 N when the blank has a cross sectional profile of about 1 mm². It is advantageous if such conducting is done at lower temperatures, because favorable mechanical properties are observed.

The method according to the invention is particularly advantageous if a further process step is foreseen and thus, without the additional rollers, there is a risk that upsetting may occur due to the interaction of this further step and the forming process from the blank to the hollow cylinder caused by the relative movement of the blank, punch and recipient towards the die.

In this context, it is particularly worth to have an additional step (iii) in which the hollow cylinder is pulled by a pulling device. Said device grips the end of the manufactured hollow cylinder facing away from the punch and pulls it in the feed direction. This improves the obtained hollow cylinder geometry, in particular the straightness of the resulting tube.

This pulling can take place with or without simultaneous torsion, whereby the additional torsion further increases the internal stress in the semi-finished product.

Advantageously, the drawing device draws the hollow cylinder through a drawing ring which encloses the cylinder and thereby, additionally smoothens the outer surface and/or reduces the diameter of the tube.

The diameter reduction by drawing is only very small at this point and amounts to only a few hundredths of a millimeter in the production of a stent. Therefore, the use of a pin, also called inner free-floating mandrel, to support the inside of the tube during drawing is not necessarily required, however, its use has proven to be advantageous, especially in the area of a drawing ring. By using a pin, the inner shell of the hollow cylinder can also be smoothened, and a possible reduction of the outer diameter is facilitated.

Alternatively or in addition to the use of a drawing ring, a machining tool can be used through which or along which the hollow cylinder is drawn by the drawing device and thus, material of the cylinder is removed. In particular turning, but also milling or grinding are suitable processes that can be used for shaping.

It is particularly advantageous if both a drawing ring and at least one machining tool are used, as this means that even more complex geometries can be produced in just a few steps.

Preferably, the blank is heated together with the punch in the recipient in a range of 200 to 300° C., and in some cases 250 to 280° C. This enables a more homogeneous forming with regard to the grain size distribution.

The advantages of the invention are particularly effective if the process, and in particular the succession of steps (i), (ii) and (iii), are carried out continuously by a directly shifting the resulting hollow cylinder, since the advantages of the individual processes regarding the microstructure are not negatively influenced by intermediate processing pauses.

This process is advantageously carried out in an apparatus designed for this purpose. Firstly, such an apparatus for producing a hollow cylinder for a medical application or for a medical product, in particular for an implant, includes an extrusion device. All features of the process described should also apply to their formation in the apparatus according to the invention and vice versa.

Such an extrusion device can receive a blank, optionally with a drill hole, in a recipient chamber of the recipient. The extrusion device further includes a punch featuring a press disc and a mandrel, both configured to be also inserted at least partly into the recipient chamber. Moreover, the extrusion device includes a die, also called matrix. The punch is arranged in such a way that its press disc can press the blank with the end face through a die in the feed direction or that the punch together with the die is pressed into the recipient so that a hollow cylinder is formed.

Secondly, at least two rollers are provided downstream of the extrusion press device in the feed direction. These rollers delimit a roll gap through which the hollow cylinder is guided. Thereby, a supporting tension can be applied to the material in the described manner, which minimizes or even prevents material upsetting or compression and improves the mechanical properties of the hollow cylinder.

Advantageously, the mechanical properties can be further improved if a pulling device (also called drawing device) is provided downstream of the rollers in the feed direction, which at least partially encloses the tube. It is particularly advantageous if a drawing ring is arranged between the rollers and the pulling device, which surrounds the hollow cylinder and additionally smoothens the outer surface of the hollow cylinder.

In addition or alternatively, at least one machining tool can be provided after the rollers and/or before or after the drawing ring. This allows to further adapt the geometric properties of the hollow cylinder.

This can be done, for example, by moving or swivelling out. A buckling of the hollow cylinders, which in the case of stent production are very small in their dimensions both in terms of external and internal diameter as well as in terms of wall thickness, can thus be prevented.

In summary, the process and the associated device provide the possibility of retaining very good mechanical properties despite high degrees of forming. Additionally, these mechanical properties can be further improved by supported tensile or torsional stress.

An optimization of the geometrical properties, in particular with regard to the possibility of reducing the outer diameter and wall thickness variations, is achieved above all by drawing through the drawing ring. The drawing process through the drawing ring simultaneously enables a hardening of the tube surface or the edge layer for the realization of better mechanical properties as a result of the applied pressure.

Particular for indirect extrusion processes and especially for relatively long dies, it has proven to be advantageous to support the dies in order to prevent them from bending or even breaking. This is achieved by at least one support device for the die. In order to enable complete movement of the die without it being hindered by the support function, a particularly advantageous design provides that this at least one support device can be moved in such a way that, when the die is approached, it moves out of its position. so that the matrix can be moved completely towards the recipient.

In addition to the improved mechanical and geometric properties of the hollow cylinders produced in this way, a significant increase in production efficiency is also noted. On the one hand, this is due to the reduction of the number of work steps compared to pure drawing process, but at the same time, the proportion of rejects due to variations in the outer diameter and wall thickness can be significantly minimized, which means that smaller production tolerances are possible. The latter also improves the quality of all hollow cylinders produced. This applies in particular to the production of stents, which have to meet special quality requirements.

As already described, the use of stents produced with such a method can widen constricted areas in the vessels. However, such an extraneously body in the body carries the risk of a gradual ingrowth of the stent, which can lead to a de novo vessel occlusion in the worst case.

One approach to solving this problem is to make implants and especially stents from a biodegradable material. Biodegradation is understood to be the result of hydrolytic, enzymatic and other metabolic degradation processes in the living organism, which are mainly caused by the body fluids coming into contact with the biodegradable material of the implant and lead to a gradual dissolution of the structures of the implant including the biodegradable material. The implant loses its mechanical integrity at a certain point due to this process. The term biocorrosion is often used synonymously with the term biodegradation. The term bioresorption additionally includes the subsequent resorption of the degradation products by the living organism.

Materials suitable for the body of biodegradable implants may contain, for example, polymers or metals. Biodegradable magnesium implants, in particular magnesium stents, have proven to be particularly promising for the above-mentioned target corridor of degradation.

In addition to any semi-finished product or hollow cylinder obtained by the process as according to the invention, the present invention particularly protects the implementation of the process using a magnesium alloy and a hollow cylinder made of a magnesium alloy.

Further objectives, features, advantages and possible applications of the invention can also be taken from the following description of the attached figure and the example. All features described and/or illustrated form the subject matter of the invention per se or in any combination, independent of their inclusion in the individual claims or their back-references.

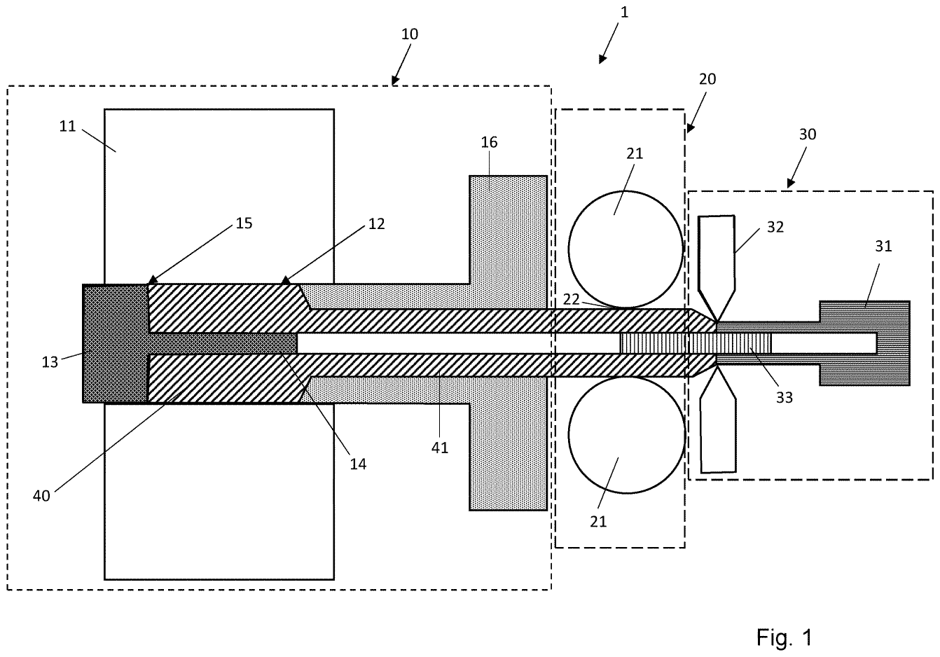

FIG. 1 shows an apparatus 1 according to the invention. This apparatus features an extrusion press device 10 including a recipient 11, a punch 13 and a die 16.

A recipient chamber 12 is provided in the recipient 11. The recipient chamber 12 is designed in such a way that a punch 13 and a blank 40 can be inserted at least partially. The punch 13 is designed in such a way that it has a press disc 15 on one side with which it can press on the blank 40. Moreover, a mandrel 14 is provided on this press disc 15, which can at least partially penetrate the blank 40.

During the pressing process, the die 16 and the punch 13 are moved relative to each other, either by moving the punch with the press disc 15 towards the die 16 or by moving the die 16 in the opposite direction into the recipient chamber 12. This deforms the blank 40 in such a way that its outer diameter assumes the round shape defined by the die 16 and, at the same time, the mandrel 14 penetrates its interior in such a way that a cylindrical cavity is created. The forming thus produces a hollow cylinder 41, featuring an outer diameter determined by the die 16 and an inner diameter determined by the mandrel 14.

Following this extrusion press device 10, there is a roller device 20 with at least two rollers 21 which define a rolling gap through which the hollow cylinder 41 is guided and thus optimized with regard to its mechanical and geometric properties.

Optionally, a further tool can be located downstream as an optimization device 30. In the variant shown, this is a pulling device 31 which draws the hollow cylinder 41 and optionally also twists it. For smoothing the outside, it can be drawn through a drawing ring 32. Smoothing and/or stabilization of the inside can be achieved by a pin 33.

In principle, the use of at least one machining tool is also possible as a supplement or alternative to this illustration.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

List of reference numbers

| | |
|---|---|
| 1 | apparatus according to the invention |
| 10 | extrusion press device |
| 11 | recipient |
| 12 | recipient chamber |
| 13 | punch |
| 14 | mandrel |
| 15 | press disc |
| 16 | die |
| 20 | roller device |
| 21 | roller |
| 22 | roll gap |
| 30 | optimization device |
| 31 | pulling device |
| 32 | drawing ring |
| 33 | pin |

1. A process for producing a hollow cylinder for an implant, comprising:
extruding a blank that is at least partially inserted into a recipient chamber of a recipient by pressing a punch through a die or by pressing both the punch and the die both into the recipient;
driving a mandrel into the blank to form a hollow cylinder with an inside diameter defined by the mandrel and an outside diameter defined by the die; and
guiding the hollow cylinder through a roll gap defined by at least two rollers.

2. The process according to claim 1, comprising pulling the hollow cylinder with a pulling device.

3. The process according to claim 2, wherein the pulling is carried out with simultaneous torsion.

4. The process according to claim 2, wherein the pulling comprising pulling the hollow cylinder through a drawing ring surrounding it to smooth the hollow cylinder.

5. The process according to claim 4, comprising introducing a pin to support the hollow cylinder from inside during the pulling.

6. The process according to claim 2, wherein the pulling draws the hollow cylinder is through a machining tool to remove material.

7. The process according to claim 4, wherein the pulling draws the hollow cylinder through a drawing ring and a machining tool.

8. The process according to claim 1, wherein the extruding, driving and guiding follow each other in a continuous process.

9. An apparatus for producing a hollow cylinder an implant, comprising:
an extrusion press device comprising a recipient with a recipient chamber configured to receive a blank;
a punch configured to be guided in the recipient chamber;
a press disc on the punch;
a mandrel on the press disc, the mandrel being configured to form an inner diameter of the hollow cylinder;
a die configured to form an outer diameter of the hollow cylinder; and
at least two rollers downstream of the extrusion press device, the at least two rollers delimiting a rolling gap configured to guide the hollow cylinder.

10. The apparatus according to claim 9, comprising a pulling device downstream of the at least two rollers, the pulling device being configured to at least partially surround the hollow cylinder and to pull it through a drawing ring to smooth the hollow cylinder.

11. The apparatus according to claim 9, comprising at least one support device provided between the recipient and the die, the at least one support device being configured to be removed when the punch disc and the die come closer together.

12. (canceled)

13. The apparatus according to claim 9, comprising a machining tool downstream of the at least two rollers.

14. The apparatus according to claim 13, comprising a pulling device downstream of the at least two rollers, the pulling device being configured to at least partially surround the hollow cylinder and to pull it through a drawing ring.
